(19) United States
(12) Patent Application Publication
(10) Pub. No.: US 2007/0238716 A1
(43) Pub. Date: Oct. 11, 2007

(54) STATIN STABILIZING DOSAGE FORMULATIONS

(76) Inventors: Ayanampudi Sri Rama Murthy, Hyderabad (IN); Korlapati Venkateswara Rao, Visakhapatnam (IN); Ranadeep Bokalial, Guwahati (IN); Ritu Kaushik, Ambala (IN); Suchitra Kaushik, Hardwar (IN)

Correspondence Address: SHERIDAN ROSS PC, 1560 BROADWAY, SUITE 1200, DENVER, CO, 80202, US

(21) Appl. No.: 11/686,284

(22) Filed: Mar. 14, 2007

(30) Foreign Application Priority Data
Mar. 14, 2006 (IN) 700/DEL/2006

(51) Int. Cl. A61K 31/555; A61K 31/401

(52) U.S. Cl. 514/184; 514/423

(57) ABSTRACT

The present invention relates to pharmaceutical formulations containing 7-[2-(4-fluorophenyl)-5-isopropyl-3-phenyl-4-[(4-hydroxy methylphenylamino) carbonyl]-pyrrol-1-yl]-3,5-dihydroxy-heptanoic acid, or pharmaceutically acceptable salts thereof, and a stabilizing agent.

### CROSS REFERENCE TO RELATED APPLICATION

This application claims the benefit of priority under 35 U.S.C. § 119 to Indian Patent Application No. 700/DEL/2006 filed Mar. 14, 2006, which is incorporated herein by reference.

### FIELD OF THE INVENTION

The present invention relates to stabilized dosage formulations containing 7-[2-(4-fluorophenyl)-5-isopropyl-3-phenyl-4-[(4-hydroxy methylphenylamino) carbonyl]-pyrrol-1-yl]-3,5-dihydroxy-heptanoic acid, or pharmaceutically acceptable salts thereof, and processes for the preparation and administration of these formulations.

### BACKGROUND OF THE INVENTION

HMG-CoA reductase inhibiting drugs, commonly referred to as “statins,” are considered a first-line therapy for the treatment of elevated cholesterol in mammals. HMG-CoA reductase is an enzyme involved in cholesterol production in the body, and statin drugs inhibit this enzyme, thereby reducing the amount and frequency of cholesterol production. These drugs are effective in the treatment of a wide variety of disease states including arteriosclerosis, atherosclerosis, ischemia, hyperlipidaemia, hyperlipoproteinemia, hypercholesterolemia, hypertriglyceridemia, hypertension, stroke, endothelium dysfunctions, peripheral vascular disease, peripheral arterial disease, coronary heart disease, myocardial dysfunction, cerebral infarction, myocardial microvascular disease, dementia, macular degeneration, kidney disease, multiple sclerosis, Alzheimer's disease, osteoporosis and/or osteopenia, angina and restenosis.

7-[2-(4-fluorophenyl)-5-isopropyl-3-phenyl-4-[(4-hydroxy methylphenylamino) carbonyl]-pyrrol-1-yl]-3,5-dihydroxy-heptanoic acid is a HMG CoA reductase inhibitor used to reduce cholesterol production in mammals in need of such treatment. It is disclosed in PCT Application No. PCT/IB2004/001761 (Publication No. WO 04/106299), which is incorporated herein, by reference. The chemical structure of this compound is shown in Formula I. A particularly preferred enantiomer of this statin drug is (3R,5R)-7-[2-(4-fluorophenyl)-5-isopropyl-3-phenyl-4-[(4-hydroxy methylphenylamino) carbonyl]-pyrrol-1-yl]-3,5-dihydroxy-heptanoic acid.

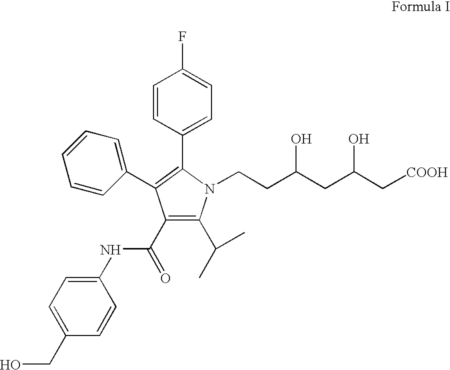

HMG-CoA reductase inhibitors are typically prepared as salts in the production of pharmaceutical dosage formulations. Unfortunately, these salts are particularly sensitive to an acidic environment, and the hydroxy acids quickly degrade to a lactone under acidic conditions. Thus, the primary instability of 7-[2-(4-fluorophenyl)-5-isopropyl-3-phenyl-4-[(4-hydroxy methylphenylamino) carbonyl]-pyrrol-1-yl]-3,5-dihydroxy-heptanoic acid is due to the extreme lability of the β, δ-hydroxy groups on the heptanoic acid chain.

PCT publication WO 00/35425 describes methods of stabilizing certain statin drugs with buffers such as sodium and potassium citrate, sodium phosphate, disodium phosphate, calcium carbonate, calcium hydrogen phosphate, calcium phosphate, calcium sulfate, sodium or magnesium carbonate, sodium ascorbinate, benzoate, sodium or potassium hydrogen carbonate, sodium or potassium lauryl sulfate and mixtures thereof. Similarly, PCT publication WO 01/93860 discloses a pharmaceutical composition containing a homogeneous mixture of a HMG-CoA reductase inhibitor with a buffering substance or a basifying substance obtained by co-crystallization and/or co-precipitation of the HMG-CoA reductase inhibitor and the buffering substance or basifying substance.

### SUMMARY OF THE INVENTION

The present inventors have discovered that for improved stability and shelf life, the compounds of Formula I should be stabilized in an environment having a pH equal to, or greater than, about pH 4.5. Additionally, they have discovered that for stabilization and shelf life of pharmaceutical formulations containing the compounds of Formula I, the pH of the dosage formulation should be equal to, or greater than, about pH 8. These pharmaceutical formulations can therefore be stabilized by the addition of an alkalinizing agent.

One embodiment of the present invention provides a pharmaceutical formulation containing 7-[2-(4-fluorophenyl)-5-isopropyl-3-phenyl-4-[(4-hydroxy methylphenylamino) carbonyl]-pyrrol-1-yl]-3,5-dihydroxy-heptanoic acid, or pharmaceutically acceptable salts thereof, and one or more stabilizing agent(s), wherein the formulation provides a pH equal to, or greater than, about pH 4.5.

Another embodiment of the present invention provides a pharmaceutical formulation containing 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or pharmaceutically acceptable salts thereof, and one or more stabilizing agent(s), wherein the formulation provides a pH equal to, or greater than, about pH 8.0. Preferably, the pharmaceutical formulation of this embodiment provides a pH in the range of about pH 8 to about pH 13. More preferably, the pharmaceutical formulation of this embodiment provides a pH in the range of about pH 9 to about pH 12.

One preferred embodiment of the present invention provides a stabilized solid dosage form containing 7-[2-(4-fluorophenyl)-5-isopropyl-3-phenyl-4-[(4-hydroxy methylphenylamino)carbonyl]-pyrrol- 1-yl]-3,5-dihydroxy-heptanoic acid, or pharmaceutically acceptable salts thereof, and one or more stabilizing agent(s), wherein the formulation provides a pH equal to, or greater than, about pH 8.0. Preferably, the stabilized solid dosage form of this embodiment provides a pH in the range of about pH 8 to about pH 13. More preferably, the stabilized solid dosage form of this embodiment provides a pH in the range of about pH 9 to about pH 12.

One preferred embodiment of the present invention provides a stabilized solid dosage form containing (3R,5R)-7-[2-(4-fluorophenyl)-5-isopropyl-3-phenyl-4-[(4-hydroxy methylphenylamino) carbonyl]-pyrrol-1-yl]-3,5-dihydroxy-heptanoic acid, or pharmaceutically acceptable salts thereof, and one or more stabilizing agent(s), wherein the dosage form provides a pH equal to, or greater than, about pH 8.0. Preferably, the stabilized solid dosage form of this embodiment provides a pH in the range of about pH 8 to about pH 13. More preferably, the stabilized solid dosage form of this embodiment provides a pH in the range of about pH 9 to about pH 12.

Another embodiment of the present invention provides a dosage formulation containing 7-[2-(4-fluorophenyl)-5-isopropyl-3-phenyl-4-[(4-hydroxy methylphenylamino)carbonyl]-pyrrol-1-yl]-3,5-dihydroxy-heptanoic acid hemicalcium salt and one or more stabilizing agent(s), wherein the dosage form provides a pH equal to, or greater than, about pH 4.5, and more preferably greater than about pH 8, and more preferably within the range of about pH 9 to about pH 12.

Another embodiment of the present invention provides a stabilized solid pharmaceutical dosage formulation containing:
from about 5-50% by weight of 7-[2-(4-fluorophenyl)-5-isopropyl-3-phenyl-4-[(4-hydroxy methylphenylamino)carbonyl]-pyrrol-1-yl]-3,5-dihydroxy-heptanoic acid hemi calcium salt,
from about 0.1 to 30% by weight of at least one stabilizing agent(s)

This embodiment can further include one or more of the following components:
from about 20-80% by weight of a diluent
from about 0.1-15% by weight of a disintegrant, and
from about 0.1-15% by weight of a lubricant/glidant.

Another embodiment of the present invention provides a pharmaceutical formulation containing a liquid preparation of 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or pharmaceutically acceptable salts thereof, and one or more stabilizing agent(s), wherein the liquid formulation provides a pH equal to, or greater than, about pH 4.5, and more preferably greater than about pH 8, and more preferably within the range of about pH 9 to about pH 12. The liquid may be formulated for delivery as a bulk liquid from which an aliquot is drawn for administration or the liquid may be incorporated into a unit dosage formulation containing the liquid such as a capsule containing the liquid. The liquids may be formulated as a solution or a suspension in an aqueous or non-aqueous liquid, or an oil-in-water or water-in-oil liquid emulsions, or as an elixir or syrup.

Another embodiment of the present invention provides processes for preparing pharmaceutical dosage formulations containing 7-[2-(4-fluorophenyl)-5-isopropyl-3-phenyl-4-[(4-hydroxymethylphenylamino)carbonyl]-pyrrol-1-yl]-3,5-dihydroxy-heptanoic acid, or pharmaceutically acceptable salts thereof, and one or more stabilizing agent(s), to produce a dosage formulation that provides a pH greater than about pH 4.5, and more preferably greater than about pH 8, and more preferably within the range of about pH 9 to about pH 12.

Another embodiment of the invention provides a method of treating or preventing hypercholesterolemia in a mammal by administering to the mammal an effective amount of 7-[2-(4-fluorophenyl)-5-isopropyl-3-phenyl-4-[(4-hydroxymethylphenylamino) carbonyl]-pyrrol-1-yl]-3,5-dihydroxy-heptanoic acid, or pharmaceutically acceptable salts thereof, in a pharmaceutical formulation containing one or more of stabilizing agent(s), wherein the pharmaceutical formulation provides a pH equal to, or greater than about pH 4.5, and more preferably greater than about pH 8, and more preferably within the range of about pH 9 to about pH 12.

### DETAILED DESCRIPTION OF THE INVENTION

The present invention is drawn to pharmaceutical dosage formulations containing 7-[2-(4-fluorophenyl)-5-isopropyl-3-phenyl-4-[(4-hydroxymethylphenylamino) carbonyl]-pyrrol-1-yl]-3,5-dihydroxy-heptanoic acid, or pharmaceutically acceptable salts thereof, having improved stability, as well as methods of making these formulations and administering these formulations for therapeutic purposes.

The present invention recognizes that 7-[2-(4-fluorophenyl)-5-isopropyl-3-phenyl-4-[(4-hydroxymethylphenylamino)carbonyl]-pyrrol-1-yl]-3,5-dihydroxy-heptanoic acid has increased stability at a pH equal to, or greater than about pH 4.5, and more particularly at a pH in the range of about pH 4.5 to about pH 13. The present invention further recognizes that pharmaceutical dosage formulations comprising 7-[2-(4-fluorophenyl)-5-isopropyl-3-phenyl-4-[(4-hydroxymethylphenylamino) carbonyl]-pyrrol-1-yl]-3,5-dihydroxy-heptanoic acid salts have improved stability when the pH of the formulation is preferably greater than, or equal to, about pH 8, and more particularly in the pH range of about pH 8 to about pH 13, and more preferably in the pH range of about pH 9 to about pH 12. The surprising stability effect for the pharmaceutical dosage formulation that is observed within the smaller subset pH range of about pH 8 to about pH 13 compared to the solution state stability of 7-[2-(4-fluorophenyl)-5-isopropyl-3-phenyl-4-[(4-hydroxymethylphenylamino) carbonyl]-pyrrol-1-yl]-3,5-dihydroxy-heptanoic acid, may be due to the presence of inert excipients in the dosage formulation, which could be affecting the stability of the dosage form, or due to unit operations followed during manufacture of the dosage formulation.

The term ‘solid dosage formulation’ as used herein includes tablets, capsules, pills and like and may be present as conventional or extended-release compositions. The term ‘liquid formulation’ as used herein includes solutions and suspensions in an aqueous or non-aqueous liquid and oil-in-water or water-in-oil liquid emulsions, which can be an elixir or syrup.

As used herein, 7-[2-(4-fluorophenyl)-5-isopropyl-3-phenyl-4-[(4-hydroxy methylphenylamino) carbonyl]-pyrrol-1-yl]-3,5-dihydroxy-heptanoic acid includes the pharmaceutically acceptable salts, solvates, enantiomers, tautomers, racemates, diastereomers, metabolites, prodrugs, and N-oxides in the ester forms, e.g. lactone forms.

The term ‘pharmaceutically acceptable salts’ refer to salts prepared from pharmaceutically acceptable monovalent, divalent or trivalent, non-toxic, metal or organic bases. Examples of such metal salts include, but are not limited to, sodium, calcium, potassium or ammonium, lithium, magnesium, zinc, aluminum, amino acid, monoalkyl ammonium, dialkyl ammonium, trialkyl ammonium, N-methyl glucamine, and preferably the hemi calcium salt.

The compound of Formula I may exist in any of the solid-state forms available, such as amorphous, crystalline or any other polymorphic form, and preferably the amorphous form. The compound of Formula I may be present in an amount within the range of from about 1% to about 60%, and preferably from about 5% to about 50%, by weight, of any of the dosage formulations described herein. In other embodiments, the compound of Formula I may be present in a range having a lower limit of about 1% by weight, about 2%, about 3% and so on in integer values up to about 20%. In such ranges, the range can have an upper limit of about 60% by weight, about 59%, about 58% and so on in integer values down to about 15%.

The phrase ‘provides a pH’ or ‘providing a pH’ with reference to a solid dosage formulation of the present invention, means that the desired pH can be determined by measuring the pH of an aqueous dispersion created by dispersing a solid unit dosage form of the formulation containing the compound of Formula I in 100 ml of water, at 25° C.

As used herein, a ‘stabilizing agent’ includes at least one alkalinizing agent, antioxidant or chelating agent. In some embodiments, stabilizing agents used in the compositions of the present invention include combinations of these agents. Suitable alkalinizing agents include alkali metal salts and alkaline earth metal salts. The alkali metal salts can include potassium bicarbonate, sodium carbonate, sodium hydroxide, sodium silicate, disodium hydrogen orthophosphate, sodium aluminate, and other suitable alkali metal salts or mixtures thereof. Suitable alkaline metal salts include calcium carbonate, calcium hydroxide, magnesium carbonate, magnesium hydroxide, magnesium silicate, magnesium aluminate, aluminum magnesium hydroxide or mixture thereof. More particularly, calcium carbonate, potassium bicarbonate, calcium hydroxide, and/or sodium carbonate may be used as alkalinizing agents. Alkalinizing agents, as described herein, can be used to obtain a formulation pH within the desired pH ranges, as described herein. The alkalinizing agent may be present in an amount within the range of from about 0.1% to about 30%, by weight, and more preferably from about 12.5% to about 30%, by weight, of the total weight of any of the dosage formulations described herein. In other embodiments, the alkalinizing agent may be present in a range having a lower limit of about 0.1% by weight, about 0.2%, about 0.3% and so on in values of one tenth percent up to 1%, and about 1% by weight, about 2%, about 3% and so on in integer values up to about 15%. In such ranges, the range can have an upper limit of about 30% by weight, about 29%, about 28% and so on in integer values down to about 15%.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration sufficient to improve the stability of the 7-[2-(4-fluorophenyl)-5-isopropyl-3-phenyl-4-[(4-hydroxy methylphenylamino) carbonyl]-pyrrol-1-yl]-3,5-dihydroxy-heptanoic acid in the formulation, and more preferably between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration sufficient to improve the stability of the 7-[2-(4-fluorophenyl)-5-isopropyl-3-phenyl-4-[(4-hydroxy methylphenylamino) carbonyl]-pyrrol-1-yl]-3,5-dihydroxy-heptanoic acid in the formulation, and more preferably between about 0.001% and about 5%, by weight, of the dosage formulation.

The term ‘stabilized’ as used herein means that a composition or formulation of the present invention maintains a content of 7-[2-(4-fluorophenyl)-5-isopropyl-3-phenyl-4-[(4-hydroxy methylphenylamino) carbonyl]-pyrrol-1-yl]-3,5-dihydroxy-heptanoic acid throughout the intended shelf life of the composition or formulation to be therapeutically acceptable. More particularly, ‘stabilized’ refers to a composition or formulation, that when subjected to an assay to determine the content of the compound of Formula I is not less than about 50% w/w throughout the intended shelf life of the formulation, not less than about 55%, not less than about 60%, not less than about 65%, not less than about 70%, not less than about 75%, riot less than about 80%, not less than about 85%, not less than about 90%, not less than about 95%, or not less than about 99%.

The dosage formulations provided by this invention may contain the compounds of Formula I, either alone or in combination with other therapeutically active ingredients, and pharmaceutically acceptable inert excipients. The term ‘pharmaceutically acceptable inert excipient’ includes at least one of diluents, binders, lubricants/glidants, coloring agents and release modifying polymers.

The dosage formulations of this invention may include one or more diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

The dosage formulations of this invention may include one or more binders within the range of from about 1% to about 60% w/w. Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscannellose sodium, crospovidone, low substituted hydroxypropyl cellulose, and the like. The concentration of a disintegrant in a dosage formulation of this invention may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration of a lubricant or glidant in a dosage formulation of this invention may vary from 0.1% to 15%, by weight, of the dosage form.

Release modifying polymers may be used to form extended release formulations containing the compounds of Formula I. The release modifying polymers may be either water-soluble polymers, or water insoluble polymers. Examples of water-soluble polymers include polyvinylpyrrolidone, hydroxy propylcellulose, hydroxypropyl methylcellulose, vinyl acetate copolymers, polyethylene oxide, polysaccharides (such as alginate, xanthan gum, etc.), methylcellulose and mixtures thereof. Examples of water-insoluble polymers include acrylates such as methacrylates, acrylic acid copolymers; cellulose derivatives such as ethylcellulose or cellulose acetate; polyethylene, and high molecular weight polyvinyl alcohols.

Coloring agents may be selected from the FDA approved colorants including, for example, Iron oxide, Lake of Tartrazine, Allura red, Lake of Quinoline yellow, Lake of Erythrosine.

Liquid dosage forms for oral administration of the compounds of the invention include pharmaceutically-acceptable emulsions, microemulsions, solutions, suspensions, syrups and elixirs. In addition to the active ingredient(s), the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. In these liquid formulations, the 7-[2-(4-fluorophenyl)-5-isopropyl-3-phenyl-4-[(4-hydroxymethylphenylamino) carbonyl]-pyrrol-1-yl]-3,5-dihydroxy-heptanoic acid may be present in an amount within the range of from about 0.1% to about 30%, weight per volume (w/v), and more preferably from about 12.5% to about 30%, w/v, of the total volume of any of the dosage formulations described herein. In other embodiments, the 7-[2-(4-fluorophenyl)-5-isopropyl-3-phenyl-4-[(4-hydroxymethylphenylamino) carbonyl]-pyrrol-1-yl]-3,5-dihydroxy-heptanoic acid may be present in the liquid formulation in a concentration range having a lower limit of about 0.1% w/v, about 0.2% w/v, about 0.3% w/v, and so on in values of one tenth percent up to 1% w/v, and about 1% w/v, about 2% w/v, about 3% w/v and so on, in integer values up to about 15% w/v. In such ranges, the range can have an upper limit of about 30% w/v, about 29% w/v, about 28% w/v, and so on in integer values down to about 15% w/v. In contrast to the solid dosage formulations of the present invention, the phrase ‘has a pH’ or ‘having a pH’ is used in reference to a liquid dosage formulation of the present invention, and means that the desired pH can be determined by measuring the pH of an aliquot of the liquid formulation containing the compound of Formula I, at 25° C.

Suspensions, in addition to the active compound, may contain suspending agents, such as ethoxylated isostearyl alcohols, polyoxyethylene sorbitol and sorbitan esters, microcrystalline cellulose, aluminum metahydroxide, bentonite, agar-agar and tragacanth, and mixtures thereof.

According to one embodiment, a solid dosage formulation is prepared by:
i) blending a compound of Formula I or pharmaceutically acceptable salts thereof with a stabilizing agent and pharmaceutically acceptable inert excipients;
ii) optionally granulating or comilling the above blend;
iii) optionally blending the granules with pharmaceutically acceptable inert extragramilar excipients;
iv) lubricating the granules/blend;
v) compressing the lubricated granules/blend into suitably sized tablets or filling into capsules.

According to another embodiment, a solid dosage formulation is prepared using a wet granulation technique, by:
i) blending a compound of Formula I or pharmaceutically acceptable salts thereof with a stabilizing agent and pharmaceutically acceptable inert excipients;
ii) granulating the above blend with a granulating fluid;
iii) optionally blending the granules with pharmaceutically acceptable inert extragranular excipients;
iv) lubricating the granules/blend;
v) compressing the lubricated granules into suitably sized tablets or filling into capsules.

According to another embodiment, a solid dosage formulation is prepared using a dry granulation technique, by:
i) blending a compound of Formula I or pharmaceutically acceptable salts thereof with a stabilizing agent and pharmaceutically acceptable inert excipients;
ii) granulating the above blend using slugging or roller compaction;
iii) optionally blending the granules with pharmaceutically acceptable inert extragranular excipients;
iv) lubricating the granules/blend;
v) compressing the lubricated granules into suitably sized tablets or filling into capsules.

According to another embodiment, a solid dosage formulation is prepared using direct compression, by:
i) blending a compound of Formula I or pharmaceutically acceptable salts thereof with a stabilizing agent and pharmaceutically acceptable inert excipients;
ii) lubricating the blend;
iii) compressing the blend into suitably sized tablets.

According to another embodiment, capsules may be formulated by:
i) mixing a compound of Formula I, or pharmaceutically acceptable salts thereof, with a stabilizing agent and pharmaceutically acceptable inert excipients,
ii) lubricating the blend,
iii) filling the blend into capsule shells.

According to another embodiment, capsules may also be formulated by:
i) mixing the compound of Formula I or pharmaceutically acceptable salts thereof along with a stabilizing agent and pharmaceutically acceptable inert excipients,
ii) comilling the above blend,
iii) adding the remaining excipients and then lubricating the blend,
iv) filling the blend into capsule shell.

Additional objects, advantages, and novel features of this invention will become apparent to those skilled in the art upon examination of the following examples thereof, which are not intended to be limiting.

### EXAMPLES

### Example 1

This example evaluates the solution state stability of 7-[2-(4-fluorophenyl)-5-isopropyl-3-phenyl-4-[(4-hydroxymethylphenylamino) carbonyl]-pyrrol-1-yl]-3,5-dihydroxy-heptanoic acid.

Solution state stability studies were performed on 7-[2-(4-fluorophenyl)-5-isopropyl-3-phenyl-4-[(4-hydroxymethylphenylamino) carbonyl]-pyrrol- 1-yl]-3,5-dihydroxy-heptanoic acid by solubilizing the hemi calcium salt of Formula I at a range of pH conditions and storing the solution for one or two weeks under different temperature conditions. The stability data collected are shown in Table 1.

TABLE 1
Solution state stability of the hemi calcium
salt of Formula I at various pH conditions.

| | | Storage | Drug |
|---|---|---|---|
| | Sampling | Condition | Content |
| Medium | Period | (° C.) | (mg/ml) |
| pH 2.0 | Initial | | 0.42 |
| Buffer | 1 week | 2-8 | 0.21 |
| | | 25 | 0.22 |
| | | 50 | 0.14 |
| | 2 Weeks | 2-8 | 0.19 |
| | | 25 | 0.18 |
| | | 50 | 0.09 |
| pH 4.5 | Initial | | 0.51 |
| Buffer | 1 week | 2-8 | 0.50 |
| | | 25 | 0.50 |
| | | 50 | 0.43 |
| | 2 Weeks | 2-8 | 0.50 |
| | | 25 | 0.48 |
| | | 50 | 0.38 |
| pH 5.5 | Initial | | 0.50 |
| Buffer | 1 week | 2-8 | 0.50 |
| | | 25 | 0.50 |
| | | 50 | 0.45 |
| | 2 weeks | 2-8 | 0.50 |
| | | 25 | 0.48 |
| | | 50 | 0.40 |
| pH 7.5 | Initial | | 0.50 |
| Buffer | 1 week | 2-8 | 0.50 |
| | | 25 | 0.50 |
| | | 50 | 0.45 |
| | 2 Weeks | 2-8 | 0.49 |
| | | 25 | 0.49 |
| | | 50 | 0.37 |
| pH 9.0 | Initial | | 0.49 |
| Buffer | 1 week | 2-8 | 0.49 |
| | | 25 | 0.49 |
| | | 50 | 0.45 |
| | 2 Weeks | 2-8 | 0.45 |
| | | 25 | 0.47 |
| | | 50 | 0.42 |
| pH 10.0 | Initial | | 0.49 |
| Buffer | 1 week | 2-8 | 0.49 |
| | | 25 | 0.49 |
| | | 50 | 0.45 |
| | 2 Weeks | 2-8 | 0.49 |
| | | 25 | 0.47 |
| | | 50 | 0.44 |
| pH 12.0 | Initial | | 0.49 |
| Buffer | 1 week | 2-8 | 0.49 |
| | | 25 | 0.49 |
| | | 50 | 0.45 |
| | 2 Weeks | 2-8 | 0.49 |
| | | 25 | 0.48 |
| | | 50 | 0.44 |

These data showed that 7-[2-(4-fluorophenyl)-5-isopropyl-3-phenyl-4-[(4-hydroxymethylphenylamino)carbonyl]-pyrrol-1-yl]-3,5-dihydroxy-heptanoic acid is stable at a pH equal to, or greater than 4.5, and more particularly at a pH in the range of pH 4.5 to pH 12.

### Example 2

This example illustrates the preparation of a solid dosage formulation of the present invention.

A solid dosage formulation was prepared containing the following ingredients:

| Ingredients | Quantity (mg)/Tablet |
|---|---|
| Hemi calcium salt of Formula I | 10.00 |
| Microcrystalline Cellulose | 46.70 |
| Croscarmellose Sodium | 3.50 |
| Calcium Hydroxide | 7.00 |
| L-Hydroxy propyl cellulose | 2.10 |
| Magnesium Stearate | 0.70 |
| Opadry | 2.10 |
| Purified water | q.s |
| Film coated tablet weight | 72.10 |

Formulation Process
1. Microcrystalline cellulose, Croscarmellose sodium, Calcium hydroxide and L-hydroxypropylcellulose were sifted along with the hemi calcium salt of the 3R,5R enantiomer Formula I.
2. Magnesium stearate and talc was added to the blend of step 1 and the mixture was blended.
3. The blend of step 2 was compressed into a tablet.
4. The tablet was film coated with an aqueous dispersion of Opadry.

A dispersion of the solid formulation was prepared in water and the pH was determined. The pH value for the dispersion of the dosage form was:
a) pH of 0.25% w/v dispersion: 10.00
b) pH of 0.50% w/v dispersion: 10.01
c) pH of 1.0% w/v dispersion: 10.06

### Example 3

This example illustrates the preparation of a solid dosage formulation of the present invention.

A solid dosage formulation was prepared containing the following ingredients:

| | |
|---|---|
| Ingredients | Quantity (mg)/Tablet |

Intragranular

| | |
|---|---|
| Hemi calcium salt of Formula I | 10.00 |
| Microcrystalline Cellulose | 46.35 |
| Croscarmellose Sodium | 2.10 |
| Potassium bicarbonate | 7.00 |
| L-Hydroxy propyl cellulose | 2.10 |

Extragranular

| | |
|---|---|
| Croscarmellose Sodium | 1.40 |
| Magnesium Stearate | 0.35 |
| Talc | 0.35 |
| Colloidal Silicon dioxide | 0.35 |

Coating

| | |
|---|---|
| Opadry | 2.10 |
| Purified water | q.s |
| Film coated tablet weight: | 72.10 |

Formulation Process
1. The hemi calcium salt of the 3R,5R enantiomer of Formula I was blended with microcrystalline cellulose, croscarmellose sodium, potassium bicarbonate.
2. The blend in step 1 was granulated using L-hydroxypropylcellulose binder solution and granules so obtained were dried.
3. The extragranular excipients were added to the granules in step 2 and blended.
4. The blend in step 3 was lubricated using magnesium stearate.
5. The blend of step 4 was compressed into a suitable tablet.
6. The tablet was film coated with aqueous dispersion of Opadry.

A dispersion of above formulation was prepared in water and the pH was determined. The pH value for the dispersion of the dosage form was:
a) pH of 0.25% w/v dispersion: 12.00
b) pH of 0.50% w/v dispersion: 12.06
c) pH of 1.0% w/v dispersion: 12.10

### Example 4

This example illustrates the preparation of solid dosage formulations of the present invention, and the drug release characteristics of the formulations.

Solid dosage formulations were prepared containing the following ingredients:

| | | |
|---|---|---|
| Example | Example | Example |
| 4A | 4B | 4C |

| | |
|---|---|
| Ingredients | Quantity (mg)/Tablet |

Intragranular

| | | | |
|---|---|---|---|
| Hemi calcium salt | 0.50 | 2.50 | 10.00 |
| of Formula I | | | |
| Microcrystalline | 57.25 | 80.00 | 47.75 |
| Cellulose | | | |
| Croscarmellose | 1.40 | 2.00 | 1.40 |
| Sodium | | | |
| Calcium carbonate | 7.00 | 10.00 | 7.00 |
| L-Hydroxy propyl | 2.10 | 3.00 | 2.10 |
| cellulose | | | |
| Purified water | q.s. | q.s. | q.s. |

Extragranular

| | | | |
|---|---|---|---|
| Croscarmellose | 0.70 | 1.00 | 0.70 |
| Sodium | | | |
| Magnesium | 0.35 | 0.50 | 0.35 |
| Stearate | | | |
| Colloidal Silicon | 0.35 | 0.50 | 0.35 |
| dioxide | | | |
| Talc | 0.35 | 0.50 | 0.35 |
| Total weight: | 70.00 | 100.00 | 70.00 |

Formulation Process
1. The hemi calcium salt of the 3R,5R enantiomer of Formula I was blended with microcrystalline cellulose, croscarmellose sodium, calcium carbonate.
2. The blend in step 1 was granulated using L-hydroxypropylcellulose binder solution and granules so obtained were dried.
3. The extragranular excipients were added to the granules in step 2 and blended together.
4. The blend in step 3 was lubricated using magnesium stearate.
5. The blend of step 4 was compressed into suitable tablet.

The drug release of these tablets was determined using USP apparatus 2, in a 900 ml medium-containing pH 6.8 trisodium phosphate buffer at 37±0.5° C. and 75 rpm. The results are given in table 2.

TABLE 2
Cumulative percent release of drug for Example 4

| | Example | Example | Example |
|---|---|---|---|
| Time | 4A | 4B | 4C |
| (min) | % Drug Released | | |
| 5 | 97 | 87 | 83 |
| 15 | 96 | 93 | 86 |
| 30 | 94 | 95 | 86 |
| 45 | 91 | 95 | 85 |
| 60 | 91 | 94 | 85 |

### Example 5

This example illustrates the preparation of solid dosage formulations of the present invention, and evaluates the stability characteristics of the formulations.

Four solid dosage formulations were prepared containing the following ingredients:

| | | | |
|---|---|---|---|
| Example | Example | Example | Example |
| 5A | 5B | 5C | 5D |

| | |
|---|---|
| Ingredients | Quantity (mg)/Tablet |

Intragranular

| | | | | |
|---|---|---|---|---|
| Hemi calcium | 0.50 | 2.50 | 10.00 | 40.00 |
| salt of Formula I | | | | |
| Microcrystalline | 52.35 | 73.00 | 42.85 | 171.4 |
| Cellulose PH 101 | | | | |
| Croscarmellose | 2.10 | 3.00 | 2.10 | 8.40 |
| Sodium | | | | |
| Calcium carbonate | 10.50 | 15.00 | 10.50 | 42.00 |
| L-Hydroxy propyl | 2.10 | 3.00 | 2.10 | 8.40 |
| cellulose | | | | |
| Purified water | q.s. | q.s. | q.s. | q.s. |

Extragranular

| Croscarmellose | 1.40 | 2.00 | 1.40 | 5.60 |
|---|---|---|---|---|
| Sodium | | | | |
| Magnesium | 0.35 | 0.50 | 0.35 | 1.40 |
| Stearate | | | | |
| Colloidal | 0.35 | 0.50 | 0.35 | 1.40 |
| Silicon | | | | |
| dioxide | | | | |
| Talc | 0.35 | 0.50 | 0.35 | 1.40 |
| Total weight: | 70.00 | 100.00 | 70.00 | 280.00 |
| Film coating | % W/W | | | |
| Opadry | 15 | 15 | 15 | 15 |
| Purified water | q.s. | q.s. | q.s. | q.s. |

Formulation Process
1. The hemi calcium salt of the 3R,5R enantiomer of Formula I was blended with microcrystalline cellulose, croscarmellose sodium, calcium carbonate.
2. The blend in step 1 was granulated using L-hydroxypropylcellulose and granules so obtained were dried.
3. The extragranular excipients were added to the granules in step 2 and blended.
4. The blend in step 3 was lubricated using magnesium stearate.
5. The blend of step 4 was compressed.
6. The tablet was film coated with aqueous dispersion of Opadry.
   pH Data for Tablets

A dispersion of the formulation from example 5C was prepared in water and the pH was determined. The pH value for the dispersion of the dosage form in example 5C was:
a) pH of 0.25% w/v dispersion: 9.08
b) pH of 0.50% w/v dispersion: 9.10
c) pH of 1.0% w/v dispersion: 9.21
   Stability Data for Tablets

The tablets prepared in this example were subjected to stability studies at different conditions. The residual percentages of the hemi calcium salt of Formula I in the tablets were determined and the results are shown in Table 3.

TABLE 3
Stability data of Example 5 under various conditions.

| | | % Assay | | | |
|---|---|---|---|---|---|
| | | | | Exam- | Exam- |
| Time | Stability | Example | Example | ple | ple |
| period | conditions | 5A | 5B | 5C | 5D |
| Initial | | 99.2 | 97.6 | 98.3 | 102.7 |
| 1 month | 40° C./75% RH | 95.2 | 98.8 | 100.2 | 103.7 |
| | 30° C./65% RH | 99.2 | 98.7 | 102.7 | 104.3 |
| | 25° C./60% RH | 99.2 | 98.7 | 102.7 | 104.3 |
| | 2-8° C. | 97.6 | 100.6 | 97.2 | 104.3 |
| 2 months | 40° C./75% RH | 91.8 | 92.1 | 95.1 | 98.0 |

As shown in Table 3, all the compositions were found to be stable throughout the course of the stability studies.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A solid pharmaceutical dosage formulation comprising 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or a pharmaceutically acceptable salt thereof, and at least one stabilizing agent, wherein the formulation provides a pH equal to, or greater than, pH 8.0.

2. The solid pharmaceutical dosage formulation of claim 1, wherein the solid pharmaceutical dosage formulation provides a pH within the range of pH 8 to pH 13.

3. The solid pharmaceutical dosage formulation of claim 1, wherein the solid pharmaceutical dosage formulation provides a pH within the range of pH 9 to pH 12.

4. The solid pharmaceutical dosage formulation of claim 1, wherein the solid pharmaceutical dosage formulation provides a pH of about pH 10.

5. The solid pharmaceutical dosage formulation of claim 1, wherein the pharmaceutically acceptable salt is selected from the group consisting of sodium, calcium, potassium, ammonium, lithium, magnesium, zinc, aluminium, amino acid, monoalkyl ammonium, dialkyl ammonium, trialkyl ammonium, N-methyl glucamine and the hemi calcium salt.

6. The solid pharmaceutical dosage formulation of claim 1, wherein the dosage form further comprises at least one inert excipient selected from the group consisting of diluents, binders, disintegrants, lubricants /glidants, coloring agents and release modifying agents.

7. The solid pharmaceutical dosage formulation of claim 1, wherein the stabilizing agent comprises an alkalinizing agent that is an alkali metal salt or an alkaline earth metal salt.

8. The solid pharmaceutical dosage formulation of claim 7, wherein the alkalinizing agent is selected from the group consisting of sodium carbonate, sodium hydroxide, sodium silicate, disodium hydrogen orthophosphate, sodium aluminate, calcium carbonate, calcium hydroxide, magnesium carbonate, magnesium hydroxide, magnesium silicate, magnesium aluminate, aluminium magnesium hydroxide or a mixture thereof.

9. The solid pharmaceutical dosage formulation of claim 7, wherein the alkalinizing agent is present in the concentration range of 12.5% to 30%, by weight, of the formulation.

10. The solid pharmaceutical dosage formulation of claim 1, wherein the stabilizing agent comprises a chelating agent selected from the group consisting of disodium edetate, edetic acid and citric acid.

11. The solid pharmaceutical dosage formulation of claim 10, wherein the chelating agent is present in the concentration range of 0.001% to 5%, by weight, of the formulation.

12. The solid pharmaceutical dosage formulation of claim 1, wherein the stabilizing agent comprises an antioxidant selected from the group consisting of butylated hydroxyanisole (BHA), sodium ascorbate, butylated hydroxytoluene (BHT), sodium sulfite, citric acid, malic acid, and ascorbic acid.

13. The solid pharmaceutical dosage formulation of claim 12, wherein the antioxidant is present in the concentration range of 0.001% to 5%, by weight, of the formulation.

14. The solid pharmaceutical dosage formulation of claim 1, wherein the formulation is a tablet or a capsule.

15. The solid pharmaceutical dosage formulation of claim 1, wherein the 7-[2-(4-fluorophenyl)-5-isopropyl-3-phenyl-4-[(4-hydroxy methylphenylamino)carbonyl]-pyrrol-1-yl]-3,5-dihydroxy-heptanoic acid is present as the 3R,5R enantiomer.

16. A stabilized solid pharmaceutical dosage formulation comprising:
from about 5% to 50%, by weight, of 7-[2-(4-fluorophenyl)-5-isopropyl-3-phenyl-4-[(4-hydroxy methylphenylamino)carbonyl]-pyrrol-1-yl]-3,5-dihydroxy-heptanoic acid hemi calcium salt;
from about 0.1% to 30%, by weight, of at least one stabilizing agent(s).

17. The stabilized solid pharmaceutical dosage formulation of claim 16, further comprising:
from about 20% to 80%, by weight, of a diluent;
from about 0.1% to 15%, by weight, of a disintegrant; and,
from about 0.1% to 15% by weight of a lubricant/glidant.

18. A solid pharmaceutical dosage formulation comprising 7-[2-(4-fluorophenyl)-5-isopropyl-3-phenyl-4-[(4-hydroxy methylphenylamino)carbonyl].-pyrrol-1-yl]-3,5-dihydroxy-heptanoic acid, or a pharmaceutically-acceptable salt thereof, as an active agent and a stabilizing agent, wherein the solid pharmaceutical dosage formulation retains at least 90% of the active agent after storage for 2 months at 40° C. and 75% relative humidity.

19. A liquid pharmaceutical formulation comprising 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or a pharmaceutically acceptable salt thereof, and at least one stabilizing agent, wherein the formulation has a pH equal to, or greater than, pH 4.5.

20. A process for the preparation of a stabilized solid pharmaceutical dosage formulation comprising:
blending 7-[2-(4-fluorophenyl)-5-isopropyl-3-phenyl-4-[(4-hydroxy methylphenylamino)carbonyl]-pyrrol-1-yl]-3,5-dihydroxy-heptanoic acid with at least one stabilizing agent and inert excipients to form a blended mixture;
granulating or comilling the blended mixture;
optionally blending granules with pharmaceutically acceptable inert extragranular excipients;
lubricating the blend; and,
compressing the lubricated blend into suitably sized tablets or filling into capsules.

21. The process according to claim 20, wherein the granulation is by wet granulation or by dry granulation.

22. A process for the preparation of a liquid pharmaceutical formulation comprising:
blending 7-[2-(4-fluorophenyl)-5-isopropyl-3-phenyl-4-[(4-hydroxy methylphenylamino)carbonyl]-pyrrol-1-yl]-3,5-dihydroxy-heptanoic acid with at least one stabilizing agent to form a blend; and,
mixing the blend with at least one pharmaceutically acceptable liquid diluent to form a liquid pharmaceutical formulation.

23. A method of treating or preventing hypercholesterolemia in a mammal comprising administering to the mammal an effective amount of a solid pharmaceutical dosage formulation comprising 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or a pharmaceutically acceptable salt thereof, and at least one stabilizing agent, wherein the formulation provides a pH equal to, or greater than, pH 8.0.

24. A method of treating or preventing hypercholesterolemia in a mammal comprising administering to the mammal an effective amount of a liquid pharmaceutical formulation comprising 7-[2-(4-fluorophenyl)-5-isopropyl-3-phenyl-4-[(4-hydroxy methylphenylamino)carbonyl]-pyrrol-1-yl]-3,5-dihydroxy-heptanoic acid, or a pharmaceutically acceptable salt thereof, and at least one stabilizing agent, wherein the formulation has a pH equal to, or greater than, pH 4.5.
